(19) United States
(12) Patent Application Publication
(10) Pub. No.: US 2012/0270851 A1
(43) Pub. Date: Oct. 25, 2012

(54) USE OF FERROQUINE IN THE TREATMENT OR PREVENTION OF MALARIA

(75) Inventors: Laurent FRAISSE, Balma (FR); Annie STRUXIANO, Aucamville (FR)

(73) Assignee: SANOFI, Paris (FR)

(21) Appl. No.: 13/457,849

(22) Filed: Apr. 27, 2012

Related U.S. Application Data
(63) Continuation of application No. PCT/FR2010/052331, filed on Oct. 29, 2010.

(30) Foreign Application Priority Data
Oct. 30, 2009 (FR) 0905212

(51) Int. Cl. A61K 31/555; A61P 33/06

(52) U.S. Cl. 514/187

(57) ABSTRACT

The present invention relates to the use of ferroquine or its N-demethylated metabolite or any of the pharmaceutically acceptable salts thereof for treating and/or preventing infections caused by the parasite Plasmodium vivax and, more generally, a parasite of the genus Plasmodium, the life cycle of which includes a phase of hepatic latency in the human host.

The present invention relates to the use of active agents that are useful for preventing and/or treating infections by the parasite Plasmodium vivax and, more generally, a parasite of the genus Plasmodium, whose life cycle comprises a hepatic lag phase in the human host.

More specifically, the present invention relates to the use of ferroquine or its N-demethylated metabolite for this purpose.

Malaria is one of the primary infectious causes of mortality worldwide and annually affects more than 5 000 000 people, among which 3 000 000 die each year.

This plague mainly affects sub-Saharan Africa, south-east Asia and Latin America.

Four main species of Plasmodium responsible for the transmission of malaria are generally distinguished: Plasmodium falciparum, Plasmodium vivax, Plasmodium ovale and Plasmodium malariae, the first two being the most widespread.

The parasites P. falciparum and P. vivax are distinguished from each other in terms of geographical coverage and their growth cycle in the human host.

P. vivax constitutes the plasmodium species that is the most widespread on all continents, except for sub-Saharan Africa where P. falciparum is predominant, despite the presence of P. malariae which may occasionally be the cause of up to a third of the cases of malaria in that area, and P. ovale, which is nevertheless rarer (Mendis K. et al., The Neglected Burden of Plasmodium vivax Malaria, Am. J. Trop. Med. Hyg., 2001, 64 (1-2 suppl) : 97-106).

More specifically, the parasite P. vivax is predominantly located in south-east Asia and in the Pacific, where it is responsible for 49% of malaria cases, but also, to a lesser extent, in the territories of east and south Africa. In point of fact, it is present in the Afro-Asiatic populations, especially in Kenya, in Tanzania and in the Indian Ocean islands, for instance Madagascar. Moreover, the prevalence of this species increases in South America and Central America, with 71% to 81% of the cases of malaria. It is especially found in Peru, Bolivia and French Guiana. 81% of cases of malaria are also attributable to P. vivax in the eastern Mediterranean regions and 100% in the ex-USSR countries.

The estimations concerning the geographical prevalence of this parasite vary according to the methodology used, but the one thing that is certain is that the importance of this parasite is largely underestimated.

As regards the growth of the parasite, the four abovementioned species are all transmitted via a female anopheles mosquito bite. Once inside the human host, the parasite reaches the liver cells, undergoes an asexual replication phase therein and leads to the formation of vesicles, the schizonts. The vesicles thus formed are released into the hepatic sinusoids and thereafter enter the blood circulation and spread therein a flood of young pre-erythrocytic merozoites ready to infect the red blood cells. The young pre-erythrocytic merozoites enter the red blood cells and start the erythrocytic cycle. The successive divisions of the merozoites cause rupture of the parasite-infested red blood cells. These sudden and synchronous ruptures are the cause of bouts of fever. The merozoites released into the blood circulation infect new red blood cells or erythrocytes. This is the start of the erythrocytic asexual cycle or erythrocytic schizogony. After invasion of the erythrocytes by the merozoites, the growth of the parasite begins via the ring stage, and then evolves into the trophozoite form.

Moreover, it should be noted that for the species other than P. falciparum, certain pre-erythrocytic merozoites do not reach the blood directly but rather attack new hepatocytes.

These hepatic forms, known as hypnozoites, remain in the latent state for a time that is particular to the type of strain and dependant on its environment. They maintain parasitosis in the liver for 2 or 3 years in the case of P. ovale, 3 to 5 years or more in the case of P. vivax and for the rest of the life in the case of P. malariae, before reactivating in successive waves, causing a strong fever, also known as a benign tertian fever, which is one of the forms of malaria.

With regard to this specificity, it is evident that active agents that are effective for treating infections caused by P. falciparum generally prove, on the other hand, to be insufficient in terms of efficacy with regard to infections induced by these other species, in so far as they are limited to removing the circulating forms of the parasite and do not in any way act on the quiescent forms stored in the human hepatocytes.

Now, the conventional antimalaria treatments available for treating all the parasitic infections of the genus Plasmodium are treatments whose efficacy has essentially been validated only on the species P. falciparum.

Thus, chloroquine was and remains the first-line treatment for P. vivax malaria since 1946. It is often recommended with a relay with 8-aminoquinoline, primaquine (World Health Organization, “Annex 10. Treatment of Plasmodium vivax, P. ovale and P. malariae infections” Jan. 1, 2006, Guidelines for the treatment of malaria, p. 225-239).

However, the phenomena of P. vivax resistance to chloroquine make this active agent less and less effective against this parasite.

As regards primaquine, it has major toxicity problems and causes an increased risk of hemolysis in the case of individuals deficient in glucose-6-phosphate dehydrogenase. These individuals suffering from glucose-6-phosphate dehydrogenase deficiency often originate from Africa, the Middle East, India, the Mediterranean basin or south-east Asia.

Other compounds, such as quinine, chloroquine, mefloquine and artemisinin derivatives have little or no effect on the hepatic form of the parasite.

Antifolates and atovaquone, which were initially used in combination for treating the parasite in its circulating phase, have also been acknowledged as being active on the hepatocytes. However, numerous cases of resistance to these active agents have appeared.

As regards ACT (Artemisinin-based Combination Therapy), very few studies have to date been devoted to its efficacy on P. vivax.

There are therefore no real effective treatments at the present time for treating and preventing infections by P. vivax and more generally for treating the hepatic latent hypnozoite forms, which are characteristic of relapses.

Consequently, there is a need at the present time for a preventive and curative treatment that is specific for infections caused by a parasite of the genus Plasmodium, whose life cycle includes a hepatic lag phase in the human host.

Thus, one subject of the present invention is the use of ferroquine or its N-demethylated metabolite or a pharmaceutically acceptable salt thereof for the treatment and/or prevention of infections caused by a parasite of the genus Plasmodium, whose life cycle includes a hepatic lag phase in the human host.

The infections are caused in particular by P. vivax, P. ovale or P. malariae and more particularly by P. vivax.

The present invention is thus directed towards ferroquine or its N-demethylated metabolite or a pharmaceutically acceptable salt thereof for its use for the treatment and/or prevention of infections of blood and/or liver cells infected with a parasite of the genus Plasmodium, in particular P. vivax, P. ovale or P. malariae, and more particularly P. vivax.

The present invention is also directed towards ferroquine or its N-demethylated metabolite or a pharmaceutically acceptable salt thereof for its use for treating and/or preventing infections of blood cells at the ring stage and mature trophozoite stage of parasitic growth.

A subject of the present invention is also ferroquine or its

N-demethylated metabolite or a pharmaceutically acceptable salt thereof or its use for treating, preventing and eliminating the quiescent hypnozoite forms stored in human hepatocytes, especially derived from infections caused by a parasite of the genus Plasmodium, in particular P. vivax, P. ovale or P. malariae, and more particularly P. vivax.

A subject of the present invention is also ferroquine or its N-demethylated metabolite or a pharmaceutically acceptable salt thereof, for its use for preventing relapses due to an infection caused by the parasite P. vivax.

A subject of the present invention is also ferroquine or its N-demethylated metabolite or a pharmaceutically acceptable salt thereof for its use for treating and/or preventing benign tertian fevers due to an infection caused by P. vivax.

A subject of the present invention is also ferroquine or its N-demethylated metabolite or a pharmaceutically acceptable salt thereof for its use for treating and/or preventing infections caused by P. vivax in the case of patients deficient in glucose-6-phosphate dehydrogenase.

This ferroquine and other related derivatives that differ from it as regards the substituents present on the quinoline ring are described in WO 96/35698.

Structurally, it results from the insertion of a ferrocene group into a chloroquine molecule and corresponds to a compound of the following structure:

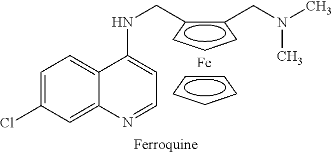

Its (N)-demethylated metabolite corresponds to a compound of the following structure:

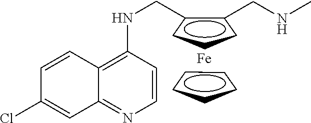

The parasitic strains specifically targeted in WO 96/35698 are the strains P. falciparum. Specifically, the development of such complexes is precisely based on the affinity of this parasite for the iron present in the red blood cells it infects.

Ferroquine is an antimalaria agent known as having higher activity than chloroquine in various parasites of the genus Plasmodium (Barends et al., “In vitro activity of ferroquine (SSR 97193) against P. falciparum isolates from the Thai-Burmese border” Malaria journal, Biomed central, vol. 6, no. 1, p. 81; Fouda et al., “On the medicinal chemistry of ferrocene” Database Biosis Biosciences information service; Olliaro et al., “The global portfolio of new antimalarial medicines under development” Clinical pharmacology and therapeutics, vol. 85, no. 6, p. 584-595).

The in vitro activity of ferroquine on chloroquine-resistant strains of P. falciparum is indicated; complementary studies are, however, necessary (Barends et al., “In vitro activity of ferroquine (SSR 97193) against P. falciparum isolates from the Thai-Burmese border” Malaria journal, Biomed central, vol. 6, No. 1, p. 81).

The present patent application demonstrates that ferroquine proves to be particularly effective for treating infections by P. vivax. Unlike chloroquine, and entirely unexpectedly, it can not only eliminate the parasite in its circulating phase in blood cells, but it also does so with equal efficacy on the immature trophozoite forms (“ring stage”) and mature trophozoite forms of P. vivax.

The active agents under consideration according to the present invention may be in free base form, but also in salt, hydrate or solvate form (the latter being defined as associations or combinations of ferroquine with, respectively, one or more water or solvent molecules).

As regards the salts that are suitable for use in the present invention, examples that may mentioned include the tartrate, L-tartrate, ditartrate, or dihydrochloride salts.

Advantageously, ferroquine is used in free base form.

A subject of the invention is also a pharmaceutical composition comprising, as active principles, ferroquine or its N-demethylated metabolite or a pharmaceutically acceptable salt thereof, for its use for treating infections by P. vivax.

Such a pharmaceutical composition contains therapeutically effective doses of ferroquine or its (N)-demethylated metabolite, or a pharmaceutically acceptable salt thereof, hydrate thereof or solvate thereof of ferroquine and also at least one pharmaceutically acceptable excipient. The said excipients are chosen, according to the pharmaceutical form and the desired mode of administration, from the usual excipients known to those skilled in the art.

The appropriate unit administration forms include oral forms such as tablets, soft or hard gel capsules, powders, granules and oral solutions or suspensions, sublingual, buccal, intratracheal, intraocular, intranasal and inhalation administration forms, topical, transdermal, subcutaneous, intramuscular or intravenous administration forms, rectal administration forms and implants. The compounds according to the invention may be used in creams, gels, ointments or lotions for topical application.

The preferred routes of administration are the oral, rectal and injectable routes.

For example, when a solid composition in tablet form is prepared, the active ingredients are mixed with one or more pharmaceutical excipients, such as gelatin, starch, lactose, magnesium stearate, talc, silica, gum arabic, mannitol, microcrystalline cellulose, hydroxypropylmethylcellulose, croscarmellose, magnesium stearate, hypromellose or the like. The tablets may be coated with sucrose, a cellulose derivative or other suitable coating materials. The tablets may be made via various techniques, such as direct tableting, dry granulation, wet granulation or hot melting.

A preparation in the form of gel capsules may also be obtained by mixing the active ingredients with a diluent and pouring the mixture obtained into soft or hard gel capsules.

Aqueous suspensions, isotonic saline solutions or sterile and injectable solutions that contain pharmacologically compatible dispersants and/or wetting agents, for example propylene glycol or butylene glycol, are used for parenteral administration.

There may be particular cases in which higher or lower doses are appropriate; such doses do not depart from the scope of the invention. According to the usual practice, the dose that is appropriate to each patient is determined by the doctor according to the mode of administration and the weight and response of the said patient.

By way of example, a unit form of administration of ferroquine in tablet form may comprise the following components:

Ferroquine 50 mg

Mannitol 224 mg

Sodium croscarmellose 6 mg

Corn starch 15 mg

Hydroxypropylmethylcellulose 2 mg

Magnesium stearate 3 mg

A subject of the present invention is also a method for treating and/or preventing malaria that includes the administration, to a patient infected with P. vivax, of a therapeutically effective dose of ferroquine or its (N)-demethylated metabolite, or a pharmaceutically acceptable salt thereof, a hydrate or a solvate thereof.

The test below is given purely as an illustration and does not in any way limit the scope of the present invention.

### In Vitro Evaluation of the Efficacy of Ferroquine Versus Other Reference Antimalaria Agents, on Blood Isolates Infected with P. vivax

The test that follows is performed on 110 isolates of P. vivax, collected in the north-west of Thailand (Tak province) on consenting patients, infected with P. vivax and in the acute phase of the infection, who came for consultation at the Shoklo Malaria Research Unit (SMRU).

The samples are collected 5 hours after their arrival and placed in 5 ml heparinized tubes, at room temperature.

The parasitemia of the collected isolates is about 4432 parasites/μl.

99 isolates are finally successfully cultured and, among these, only those containing more than 80% of trophozoites on microscopic observation are retained.

The platelets and leukocytes are removed from these isolates, by analogy with the method described in Kanlaya et al., Malaria Journal, 2009, 8:115.

About sixty samples are finally retained and ten isolates per plate or per experiment are tested in duplicate.

The activity of the various active agents is tested in predosed wells, by analogy with the method described in Barends et al., Malaria Journal 2007,6:81 and Brice et al., Antimicrobial agents and chemotherapy, January 2003, pp 170-173.

### BRIEF DESCRIPTION OF THE DRAWINGS

FIG. 1 showing a dose/response diagram illustrates the EC50 results (effective concentration 50) obtained for ferroquine and reference antimalaria agents, chloroquine, mefloquine and piperaquine.

FIG. 2 showing a dose/response diagram illustrates the IC50 results (inhibitory concentration 50) obtained for ferroquine and chloroquine.

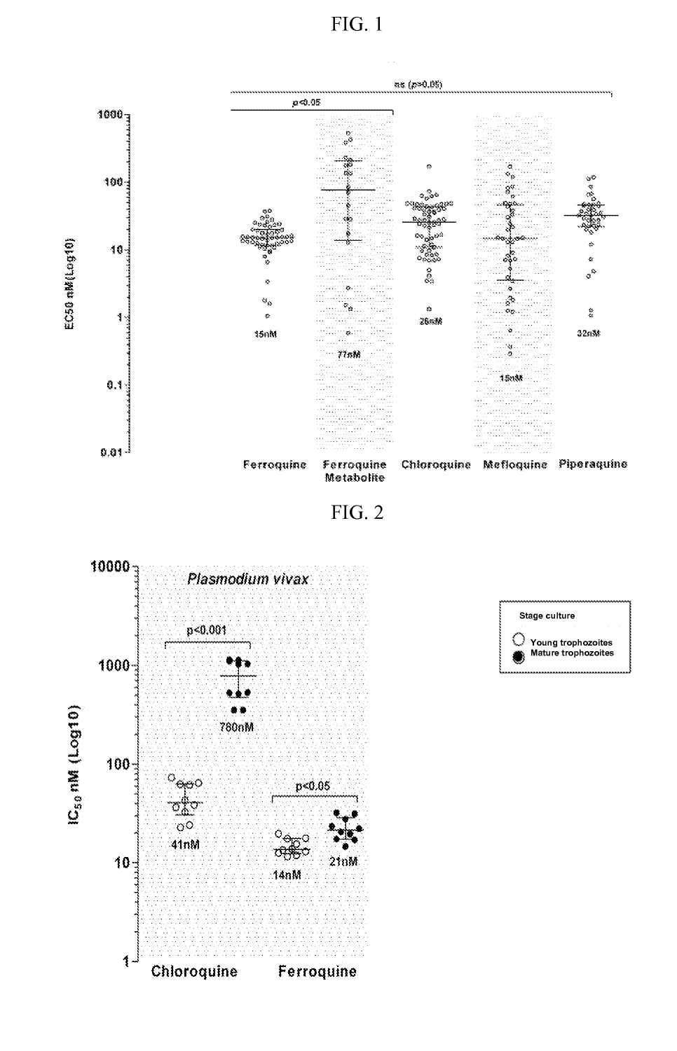

As indicated in the abovementioned FIG. 1, the lowest EC50, of about 6 nM, is that of ferroquine, thus demonstrating its increased efficacy relative to the standard antimalaria agents.

As indicated in the abovementioned FIG. 2, ferroquine is tested for its activity on the mature trophozoite stages and the young immature trophozoites (“ring stage”) of P. vivax. Chloroquine is 10 to 20 times less active on the mature trophozoite stages than on the ring stages, as also described in

Russell, B. et al. 2008, Antimicrob. Agents Chemother. 52:1040-5. On the other hand, and entirely unexpectedly, ferroquine is active on both stages of the parasite tested, with comparable activities of 14 and 21 nM for the ring and mature trophozoite stages, respectively. This demonstrates potential activity of ferroquine on various stages of growth of P. vivax, higher than that of chloroquine and thus a therapeutic advance over chloroquine.

What is claimed is:

1. A method for the treatment of infections of blood cells caused by Plasmodium vivax at the stages of parasitic growth of the immature trophozoite “ring stage” and of the mature trophozoite, the method comprising administering to a patient in need thereof an effective dose of ferroquine or its N-demethylated metabolite or a pharmaceutically acceptable salt thereof.

2. The method according to claim 1, wherein the infections are infections of the liver cells.

3. The method according to claim 1, wherein the treatment is the treatment of benign tertian fever.

4. The method according to claim 1, wherein the treatment is the treatment of infections in patients deficient in glucose-6-phosphate dehydrogenase.
